(19) United States
(12) Patent Application Publication
(10) Pub. No.: US 2007/0168037 A1
(43) Pub. Date: Jul. 19, 2007

(54) Orthopedic implant

(76) Inventor: Jeffrey C. Posnick, Potomac, MD (US)

Correspondence Address: STEPTOE & JOHNSON LLP, 1330 CONNECTICUT AVENUE, N.W., WASHINGTON, DC, 20036, US

(21) Appl. No.: 11/621,845

(22) Filed: Jan. 10, 2007

Related U.S. Application Data
(60) Provisional application No. 60/758,563, filed on Jan. 13, 2006.
(60) Provisional application No. 60/782,531, filed on Mar. 16, 2006.

(51) Int. Cl. A61F 2/44; A61F 2/30

(52) U.S. Cl. 623/17.14; 623/23.39

(57) ABSTRACT

A orthopedic implant includes an endplate having a tapered thickness, a second endplate, and a spacer disc positioned between two endplates. The disc can include polypropylene.

### CLAIM OF PRIORITY

This application claims priority under 35 USC §119(e) to Provisional U.S. Patent Application Ser. No. 60/758,563 filed on Jan. 13, 2006, and Provisional U.S. Patent Application Ser. No. 60/782,531 filed on Mar. 16, 2006, each of which is hereby incorporated by reference.

### TECHNICAL FIELD

The present invention relates to materials for orthopedic implants.

### BACKGROUND

Generally, orthopedic implants such as intervertebral discs involve the use of semi-rigid artificial joints that allow motion in one or more planes. Existing designs tend to encounter problems of improper fit, migration into cancellous bone, or dislocation. Other intervertebral implants are directed to non-rigid cushions designed to replace the nucleus pulposus of the disc, but not the intervertebral disc in its entirety. Examples of these artificial discs are described in U.S. Pat. No. 4,904,260. With nucleus pulposus replacement discs, the anulus fibrosus is typically damaged during implantation.

Most orthopedic implants require adjacent bone surfaces to be burred, drilled, or otherwise modified to keep the implant anchored to the bone surface. Burring or modifying the natural surface of a vertebral body increases the time required for surgery and also exposes a subject to additional complications and increased risk of future orthopedic damage.

### SUMMARY

Materials to be used for an orthopedic implant can include polypropylene. In one aspect, an orthopedic implant can include a first endplate having a tapered thickness; a second endplate; and a spacer disc positioned between the two endplates.

In one aspect an orthopedic implant can have a tapered thickness in a first endplate and/or a second endplate. A tapered thickness can refer to a varying thickness between the exterior surface of the endplate, and the interior surface of endplate, such that the varying thickness provides stability for the spacer disc.

The implant can include an endplate that has a rounded edge. The endplate can include titanium. The endplate can include a cobalt chromium alloy. The implant can include a spacer disc that includes at least one projection. The spacer disc can include a top surface and a bottom surface. The projection can be positioned on the top surface or the bottom surface of the spacer disc. The implant can include an endplate that includes at least one depression corresponding to at least one projection on the spacer disc.

In another aspect, an orthopedic implant can include a disc having at least two thickness values conforming to a distance between a first endplate and a second endplate, the disc including a metal ring, a first endplate having a first tapered thickness, and a second endplate having a second tapered thickness.

In certain circumstances, an orthopedic implant can include a first endplate having a fixation element on an exterior surface and a concave interior surface, a second endplate having a fixation element on an exterior surface and a concave interior surface, and a convex polypropylene disc molded to a volume that conforms to the space between the interior surfaces of the first and second endplates.

In other circumstances, a method of manufacturing an orthopedic polypropylene implant can include obtaining a molded disc, positioning the disc on a first endplate, and positioning a second endplate over the disc.

In other circumstances, a method of placing a surgical implant in a subject can include obtaining an orthopedic implant, contacting a first endplate with a natural surface of a first vertebra, and contacting a second endplate with a natural surface of a second vertebra, the first endplate and the second endplate being separated by a spacer disc.

The orthopedic implant described herein can be capable of being implanted while maintaining the natural surface area of the vertebrae after surgery. The implant can have a tapered thickness. The implant can include polypropylene. The advantage of polypropylene is that it can maintain its shape in temperatures as high as 104° C. Furthermore, a molded polypropylene implant can have stiffness similar to that of bone material. The implant can have a bone-like feel, which gives it the durability and versatility for shaping.

### DESCRIPTION OF DRAWINGS

FIG. 1 is a diagram depicting a cross section of an orthopedic implant.

FIG. 2 is a diagram depicting an endplate having a tapered thickness contacting a natural surface of a vertebra.

FIG. 3 is a diagram depicting a spacer disc and an endplate.

FIG. 4 is a diagram depicting a spacer disc and an endplate.

FIG. 5 is a diagram depicting a spacer disc and an endplate.

### DETAILED DESCRIPTION

Referring to FIG. 1, an orthopedic implant 1 can include a first endplate 11 and a second endplate 12 separated by a spacer disc 13. The disc can have a mirror-image symmetry across a horizontal plane 18. One or both endplates can include a change from a first thickness value 14 to a second thickness value 20 to create a tapered or varying thickness. The first thickness value can be greater than the second thickness value, or vice versa. A tapered thickness refers to a varying thickness between a lateral portion 15 of the endplate and a medial portion 16 of the endplate. The tapered thickness can be a varied thickness of the endplate with respect to the spacer disc. For example, a tapered thickness, can refer to a concave thickness from a cross-section. The first or second endplate can have a fixation element such as a gripping projection, rib, or roughening 17 on an exterior surface 19. The exterior surface of the endplate can be a standard or substantially flat surface. The exterior surface of the endplate can also be a customized, shaped, or curved surface.

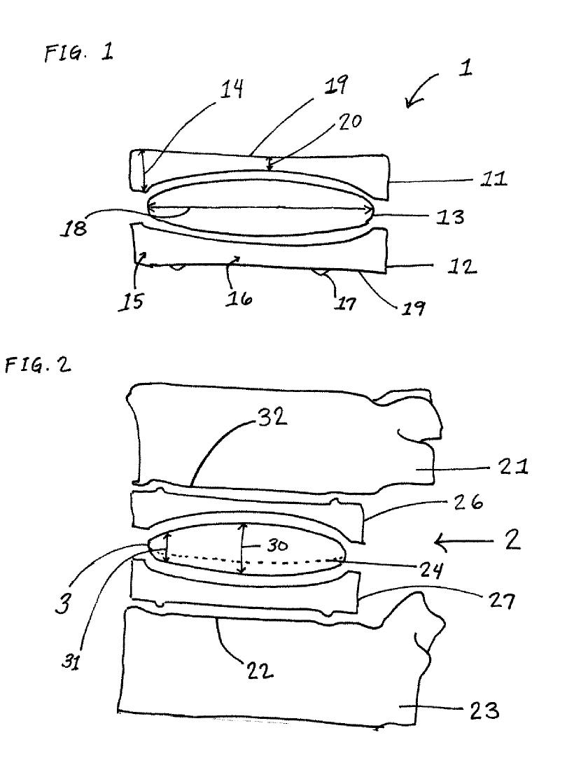

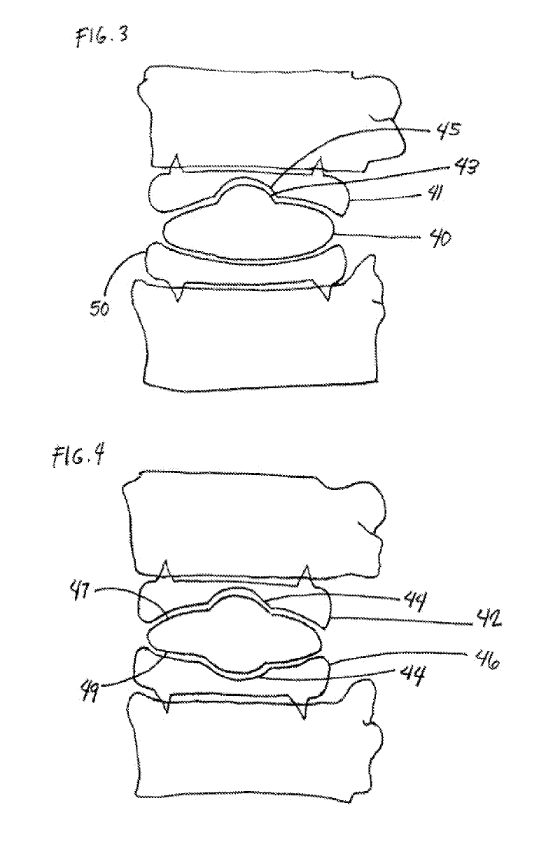

Referring to FIG. 2, an orthopedic implant 2 can include a first endplate 26 having tapered thickness that contacts a first natural surface 32 of a first vertebra 21. The implant can also include a second endplate 27 having a tapered thickness that contacts a second natural surface 22 of a second vertebra 23. The spacer disc 3 can separate the first and second endplates. The spacer disc can have at least two thickness values, such as a medial thickness 30 and a lateral thickness 31, that conform to a space between the endplates. The disc can include a metal ring 24 to allow the disc to be viewed by x-ray or other radiography techniques.

Referring to FIG. 3, and orthopedic implant can include a spacer disc 40 having at least one projection 43. The projection can be located in the medial area of the spacer disc. The projection can be positioned at the center of the spacer disc. An endplate can have at least one rounded edge 50. The rounded edge can provide a smoother fit between the endplate and an adjacent vertebra. A projection can provide an increased surface area of the disc, and a depression can provide an increased surface area of an endplate. The increased surface area can result in a greater number of contact points between the disc and an endplate. The projection can provide a better fit and minimize chances of disc slippage.

Referring to FIG. 5, a projection 43 can be located off-center or at two or more points on a spacer disc 40. An orthopedic implant can include an endplate 41 having at least one depression 45 corresponding to at least one projection on the spacer disc. An endplate can have at least one rounded edge 50. The rounded edge can provide a smoother fit between the endplate and an adjacent vertebra. Two or more projections can provide an increased surface area of the disc, and two or more depressions can provide an increased surface area of an endplate. The increased surface area can result in a greater number of contact points between the disc and an endplate. The projection can provide a better fit and minimize chances of disc slippage. Two or more projections can be equal in length, shape, or volume. Alternatively, two or more projections can be distinct in length, shape, or volume. Each projection can have a corresponding depression. A corresponding depression can have a substantially similar length, shape, or volume as its corresponding projection. A projection can have a rounded surface, a smooth surface, a rough surface, or an angular surface. A projection can include the same material as the spacer disc, or it can be composed of a different material than the spacer disc. A projection can be molded with a spacer disc in a single step. Alternatively, a projection can be added after the spacer disc has been formed or molded.

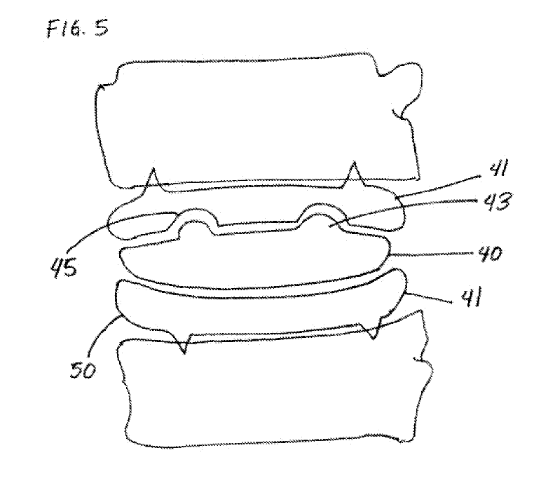

Referring to FIG. 4, the first endplate 42, the second endplate 46, or both endplates can include a depression 44. The spacer disc can have a projection on a top surface 47, a bottom surface 49, or both surfaces.

A depression and projection can be corresponding if they are substantially equal in measurement or substantially complementary in shape. For example, the height of a projection can be substantially equal to the depth of a depression, such that the projection fits into or aligns with the depression. The shape of a projection can be substantially complementary to the shape of a depression. For example, if the projection is a hemisphere shape, the depression can have a complementary hemisphere shape.

Patients or subjects with degenerative lumbar disc disease, hernia, or other vertebral pathologies, can be treated by implanting an orthopedic implant, such as an artificial intervertebral disc. Typically, orthopedic implants require bone surfaces to be burred or otherwise modified in order to keep the implant anchored to the bone surface. The disadvantages of burring or modifying the vertebral surfaces during surgery include exposing a subject to additional complications such as degenerative bone disease, increasing the time involved for surgery, and increasing the subject's post-surgical recovery period. There is a need for an orthopedic implant that can replace a natural intervertebral disc without damaging the adjacent vertebrae. A preferred implant would contact the natural surfaces of adjacent vertebrae with a precision design having a tapered thickness at the endplates. The implant can provide improved fit when contacting adjacent vertebrae such that the endplates have an enhanced stability against the natural surfaces of the adjacent vertebrae.

An orthopedic implant such as an intervertebral disc can include a first endplate and a second endplate separated by a spacer disc. An endplate can have an exterior surface that contacts an adjacent vertebra. An endplate can have an interior surface that is contoured according to the volume of a spacer disc. The endplates and the spacing piece can be manufactured from materials approved for implant engineering; for instance, the endplates are made of noncorroding metal, such as cobalt chromium or titanium alloys. See for example, U.S. Pat. No. 4,759,766, which is incorporated by reference herein. The spacing piece can be made of polypropylene of high compression and tension strengths. The reverse material combination is also possible. Other alloplastic and bioactive materials can also be used. The contact areas of the interior surface of an endplate against the spacer disc can be provided with high polish in order to minimize abrasion, utilizing a low-friction principle. The contact areas of the exterior surface of an endplate against the natural surface of a vertebra can be provided with high abrasion, using a high-friction principle. Endplates can also include annular elements, such as those described in U.S. Pat. No. 6,682,562, which is incorporated by reference herein.

One endplate can have a tapered thickness. A second endplate can have a tapered thickness. A tapered thickness can include a varying thickness between at least two thickness values. For example, an endplate can have at least one medial thickness and at least one lateral thickness. The lateral thickness can be greater than the medial thickness. Alternatively, the medial thickness can be greater than the lateral thickness. A medial thickness refers to any thickness value that is relatively more proximal to a medial axis than a lateral thickness. A medial thickness can be less than 2 mm, 4 mm, or 6 mm. A lateral thickness can be greater than 3 mm, 5 mm, or 7 mm.

The tapered thickness of the endplates can stabilize the disc in an intervertebral space and prevent dislocation. The tapered thickness can include varying thicknesses that correspond to a number of contact points between an external surface of an endplate and the adjacent vertebral body. The number of contact points can be determined mathematically and optimized so as to minimize and prevent implant dislocation. A contact point can also be supplemented by a fixation element, such as a tooth, ribbing, projection, or roughening on the exterior surface of an endplate. Bone-engaging projections are described in detail, for example, in U.S. Pat. No. 6,962,606, which is incorporated by reference herein. The fixation element can provide sufficient adherence, anchorage, friction, force or pressure, to prevent dislocation of the implant.

An endplate can be manufactured from a metal alloy, such as a cobalt chromium or titanium alloy. An endplate can have a concave interior surface for contacting a spacer disc. An endplate can have an exterior surface for contacting a natural surface of a vertebral body. An endplate can have a varying thickness that conforms to the a natural surface of a vertebral body. An endplate can include a fixation element, such as a projection, rib, or roughening on an exterior surface. The fixation element can correspond to cortical or cancellous portions of the vertebral body.

The implant can include an endplate that is elliptical in shape. The first and second endplates can be symmetrical. The first and second endplates can also be asymmetrical. For example, an inferior endplate can have a different size than a superior endplate. The difference in size of the endplates can be determined based on the location of the disc within the spine, such as a subject's lumbar curve.

The implant can include a coating that facilitates bone ingrowth, such as hydroxyapatite. The implant can also include other coatings that improve biocompatibility, such as coatings that include specialized functional groups.

The implant can include a spacer disc positioned between a first and second endplate. The disc can be a polypropylene disc. A polypropylene disc can include polypropylene and other materials or additives. The disc can rotate to provide motion in one or more planes.

The disc can be molded to a volume having a contoured shape. The disc can be a convex disc. The disc can have mirror-image symmetry across a horizontal plane or a vertical plane. The disc can be molded to conform to the space between the interior surfaces of the first and second endplate.

The disc can have a medial height that tapers toward a lateral edge of the disc. A medial height is any height that is relatively more proximal to a medial axis. A lateral height is any height that is relatively more distal from a medial axis. The disc can have a medial height greater than 15 mm, greater then 10 mm, or greater than 7 mm. The disc can have a lateral height of greater than 7 mm, greater than 5 mm or greater than 2 mm. The height of the spacer disc can vary according to the height of the space between the endplates.

The disc can be molded to a volume having rounded edges. The disc can include a metal ring or other radiopaque material to facilitate x-ray detection, or detection by other radiography techniques.

The disc can allow motion in one or more planes. The fit of the disc between the two endplates has sufficient contact area to prevent dislocation of the disc. The minimum contact area can be mathematically calculated as a function of the radius of the disc and radial curve of an endplate.

The first and second endplates can be designed to be an optimal size, which can be customized depending on the subject and the location of the vertebral disc to overcome migration of the implant into the vertebrae, especially when only the center of cancellous bone supports the implant. An optimal size of an endplate would allow an endplate to be as close to the size of adjacent vertebra, but without being too large so as to risk damaging vital organs such as the spinal cord or aorta.

The exterior surface of the endplates can be customized or designed to follow the natural surface of the individual specific adjacent vertebrae as closely as possible to avoid dislocation of the disc or implant after surgery. Generally, pins, spikes, or teeth can be positioned on the exterior surfaces of endplates, but these alone may not be sufficient to minimize risk of dislocation of the disc or implant. By providing endplates with a tapered or varying thickness, the implant can be positioned with a minimal gap between the exterior surface of endplates and the natural surface of the vertebrae and also provide sufficient contact area to prevent dislocation of the implant. To create customized implants, computer-aided design can be used, which can be based on computed tomography (CT), MRI, ultrasound, laser, and other imaging methods.

The minimum required area of contact between sagittal and transversal areas of an endplate and a vertebral body can be mathematically calculated as a function of the vertebral body size, radius of curvature of a vertebral body, and the height or area of the fixation element such as projections, teeth, or ribs on an exterior surface of an endplate. The tapered or varying thickness of the first and second endplates can anchor an disc in an intervertebral space with sufficient contact area such that the implant exerts sufficient pressure to prevent dislocation or infection. The tapered or varying thickness may decrease the size of the gap between the surface of the implant and the vertebral body. The ingrowth or healing of the vertebra to the endplate will be faster when the gap between the implant and the bone is smaller and the movement at the interface is limited, thereby decreasing the chance of dislocation. See for example, Hayashi, et al. Biomaterials 1999; 20(2): 111-9, which is incorporated by reference herein.

A method of manufacturing an orthopedic polypropylene implant can include obtaining a molded disc, positioning the disc on a first endplate, and positioning a second endplate over the disc.

A method of placing a surgical implant in a subject can include obtaining an orthopedic implant contacting a first endplate with a natural surface of a first vertebra, and contacting a second endplate with a natural surface of a second vertebra, the first endplate and the second endplate being separated by a spacer disc. Other methods of inserting a spinal implant are described in detail, for example, in U.S. Pat. No. 6,814,737, which is incorporated by reference herein.

The spacer disc can be a polypropylene disc. A polypropylene disc can include polypropylene and other materials. For example, a polypropylene disc can include polyethylene, polyamide, polyester, polycarbonate, polysulfone, polymethylmethyacrylate, hydrogels, and silicone rubber. Polypropylene can also be blended with other additives, other polymeric materials or functional additives to form a polypropylene disc. Other additives and polymeric materials that may be blended with polypropylene are disclosed in U.S. Pat. No. 5,929,129, which is incorporated by reference herein.

Polypropylene has several properties that make it advantageous as a orthopedic implant: it is thermoplastic, biocompatible, durable, inexpensive, easily shaped, and resists deformation. Polypropylene has a higher melting point (150-173° C.), higher softening point (110-170° C.), higher polymer melt index (2.0-50.0), higher tensile strength, and greater rigidity than polyethylene. It can also be less permeable than polyethylene to liquids and gases. Because of the aforementioned characteristics, polypropylene can be used as a biomaterial while requiring fewer additives compared to polyethylene.

Polypropylene can be particularly advantageous as a orthopedic implant because it has a lower density, ranging from approximately 0.880 to 0.920 grams per cubic inch, in comparison to other thermoplastic materials and high density polyethylene (HDPE), thus allowing for potential weight reductions. Polypropylene can have a high heat resistance and can be used in continuous environments as high as 220° F. (104° C.).

Polypropylene can also be highly resistant to chemical attack from solvents and chemicals in very harsh environments. In general, polypropylene is not susceptible to environmental stress cracking, and it can be exposed under load in the toughest environments. Resistance to weathering may be limited without the use of ultraviolet light absorbers, or stabilizers.

Polypropylene does not need drying prior to molding as opposed to most thermoplastic materials because polypropylenes are not hygroscopic. Therefore, a processor can work with the polypropylene material out of the container rather than having to add an initial step for drying the material. Furthermore, the excellent fatigue resistance and flexural modulus of polypropylene can make it a particularly suitable material for a surgical implant.

There are two primary types of polypropylene: homopolymer and copolymer. Homopolymer polypropylene can have a higher tensile strength than copolymer polypropylene and it is less costly. Copolymer polypropylene can have a higher impact strength but a lower tensile strength.

Unlike polyethylene, polypropylene will not polymerize via by free radical polymerization. Polypropylene can be made from the monomer propylene by Ziegler-Natta polymerization and by metallocene catalysis polymerization, or other methods, which are known in the art. Propylene can be fed to a nitrogen-blanketed reactor. The typical Ziegler-Natta catalysts, which can include TiCl₃ or TiCl₄, are used in a hydrocarbon media. Hydrocarbon solvents can be fed to the reactor. The typical temperature range of the reactor is 370° to 430° F. The reactor pressure can range from 250 to 350 psi, depending on the utilized commoners and solvents. The manufacturing process can be used as a continuous or a semicontinuous operation. After the reaction, the unreacted propylene and solvent can be removed, typically under a vacuum to ensure complete removal. Solvents can be sent to the solvent recovery system. The reaction product is chilled with water and passed through the cutter system. Companies can use different technologies to shape/pelletize the final products. Depending on the catalyst and the polymerization method used, the molecular configuration can be altered to produce various types of polypropylene, such as atactic, isotactic, syndiotactic, and elastomeric polypropylene.

Atactic polypropylene is characterized as a tacky polymer with amorphous behavior and low molecular weight. With atactic polypropylene, the pendent methyl groups are arranged randomly along the backbone of the molecule. Atactic polypropylene can be incorporated in adhesive, sealant, asphalt modification and roofing applications. Atactic polypropylene can also provide the same effect as a plasticizer, by reducing the crystallinity of the polypropylene. A small amount of atactic polymers in the final polymer can be used to improve certain mechanical properties. This can provide beneficial properties to the final polymer, such as improved low temperature performance, elongation, processability and optical properties.

Syndiotactic polypropylene can be produced in the laboratory and is manufactured, for example, by Arkema Canada, Inc. It has not been commercially used to the same degree as other forms of polypropylene.

Isotactic polypropylene has stereoregular configuration of the pendent methyl groups, and this configuration provides crystallinity in the polymer. Many of polypropylene 's mechanical properties and processability can be determined by the level of isotacticity. The increased crystallinity of polypropylene can provide a higher flexural modulus, and tensile properties much higher than polyethylene.

Elastomeric homopolypropylene has a combination molecular structure of isotactic and atactic polypropylene. This configuration can provide elasticity in the polymer and a combination of isotactic and atactic polypropylene properties.

The basic difference between polypropylene and other thermoplastic materials such as polycarbonate, polycarbonate/ABS blends and polystyrene, is that polypropylene is a semicrystalline polymer, whereas other thermoplastic materials are classified as amorphous polymers.

Due to its higher crystallinity, polypropylene has excellent moisture barrier properties and good optical properties. High crystallinity imparts improved chemical resistance in comparison to amorphous polymers. Therefore, polypropylenes can be exposed to a wide variety of agents without failure in comparison to amorphous polymers. Part shrinkage for polypropylene is higher than for amorphous polymers. This is due to better packing of the molecular chains in the crystalline regions. Differences in cooling lead to differences in crystallinity and thus differences in shrinkage. Therefore, controlling process variables, such as mold temperature and cooling time, plays a major role in determining mold shrinkage for semi-crystalline materials such as polypropylene.

Polypropylene can crystallize by forming branched structures which grow until they either exhaust the supply of crystallizing material or affect their surroundings such as to prevent further crystallization from occurring. The crystals grow by branching the degree of which depends upon temperature, chain branch structure, concentration, and nature of surrounding material (solvent or melt). At low concentration, these may interlock, forming a space-filling structure. Any remaining crystallizable polymer can then fill in the spaces between crystals within this network. Noncrystallizable material can remain within this structure. If this material is a volatile solvent, its evaporation can lead to a foam.

Polypropylene may be linear or branched. Linear polypropylene can have a relatively low level of melt strength and melt drawability. Branched propylene polymer can have a very high melt strength in combination and a relatively higher melt extensibility. With blends of linear and pure branched propylene polymers, the melt strength, melt extensibility and strain hardening behavior can increase with the amount of branched polypropylene.

The growth and morphology of polypropylene structures can be followed by the combined use of wide-angle x-ray diffraction, small-angle x-ray scattering, and small-angle light scattering. For following kinetics, synchrotron x-ray sources can be employed. In the case of foams, surface areas can be measured using gas adsorption techniques.

Polypropylene can be blended with other additives, other polymeric materials or functional additives. Other additives and polymeric materials that may be blended with polypropylene are disclosed in U.S. Pat. No. 5,929,129, which is incorporated by reference herein. Other polymeric materials can include, for example, low density polyethylene, high density polyethylene, linear low density polyethylene, medium density polyethylene, polypropylene, ethylene propylene rubber, ethylene propylene diene monomer terpolymer, polystyrene, polyvinyl chloride, polyamides, polyacrylics, cellulosics, polyesters, and polyhalocarbons. Copolymers of ethylene with propylene, isobutene, butene, hexene, octene, vinyl acetate, vinyl chloride, vinyl propionate, vinyl isobutyrate, vinyl alcohol, allyl alcohol, allyl acetate, allyl acetone, allyl benzene, allyl ether, ethyl acrylate, methyl acrylate, methyl methacrylate, acrylic acid, and methacrylic acid may also be used. Various polymers and resins which find wide application in peroxide-cured or vulcanized rubber articles may also be added, such as polychloroprene, polybutadiene, polyisoprene, poly(isobutylene), nitrile-butadiene rubber, styrene-butadiene rubber, chlorinated polyethylene, chlorosulfonated polyethylene, epichlorohydrin rubber, polyacrylates, and butyl or halo-butyl rubbers. Other resins are also possible, as will be apparent to one skilled in the art, including blends of the above materials. Any or all of the additional polymers or resins may be advantageously grafted or cross-linked, in concert or separately, within the scope of the object of this invention.

The Composition Distribution Breadth Index (CDBI) is a measurement of the uniformity of distribution of comonomer to the copolymer molecules, and is determined by the technique of Temperature Rising Elution Fractionation (TREF), as described in, for example, Wild et. al., J. Poly. Sci., Poly. Phys. Phys. Ed., Vol. 20, p. 441 (1982). This attribute relates to polymer crystallizability, optical properties, toughness and many other important performance characteristics of compositions of the present art. For example, a polyolefin resin of high density with a high CDBI would crystallize less readily than another with a lower CDBI but equal comonomer content and other characteristics, enhancing toughness in objects of the polymeric material.

Polypropylene is biocompatible and has been used successfully in the human body as a mesh for hernia repair, such as the DAVOL BARD® Mesh, which is commercially available. Medical literature, such as Law NW, Ellis H., A comparison of polypropylene mesh and expanded polytetrafluoroethylene patch for the repair of contaminated abdominal wall defects. An experimental study in Surgery 1991; 109:652-5, also shows polypropylene combined with polytetrafluoroethylene being used to repair the abdominal wall.

A orthopedic implant can be formed by obtaining polypropylene pellets, molding the pellets to a contoured shape, and fusing the pellets. A compression mold can be used to fuse the pellets, for example, by sintering. Sintering is the process of bonding of adjacent surfaces of particles, such as pellets, by heating or applying pressure. Sintering may occur with softening, without melting, with melting, or with partial melting. Polypropylene can be particularly suitable for sintering because of its relatively low melting temperature and their low thermal conductivity. Sintering is described in detail in U.S. patent application Ser. No. 60/729,728, which is incorporated by reference herein.

Polypropylene has a higher softening temperature (generally 110-170° C.) and is generally stiffer than polyethylene, which renders it more durable, versatile and suitable for further manipulation. The disadvantage of polyethylene it that it has a lower softening temperature. For example, it can soften at 82° C., which limits that amount of manipulation that can be performed on the product to create a customized shape. Ideally, an implant can be manipulated in any way to any customized shape. Orthopedic implants in particular require a significant degree of customization because the orthopedic features of every patient are unique. One method known as burring refers to forming a projecting edge by shaving the implant to a sculpted form. A burred shape refers to a sculpted form. Burring generates heat that can distort the shape of polyethylene, which has a lower softening temperature.

The advantage of polypropylene is that it can maintain its shape in temperatures as high as 100° C. Polypropylene, which has a density of approximately 0.9 g.mL, can be used to form a molded polypropylene product can have stiffness similar to that of bone material. The product can have a bone-like feel, which gives it the durability and versatility for shaping. The initial shape of the product can follow that of the mold. The initial shape can be manipulated to a customized shape based on a desired shape, implant location, or additional materials or grafts to be used.

The polymer can then be allowed to equilibrate, and can subsequently subjected to additional pressure, depending on the desired pore size. Typically, a greater pressure and a higher temperature, for longer time periods can result in a smaller pore size and greater mechanical strength. Once a porous material has been formed, the mold can be allowed to cool. If the mold was subjected to pressure, the cooling can occur while it is being applied or after it has been removed. The material can be removed and then optionally processed. Examples of processing can include, sterilizing, shaping, cutting, trimming, polishing, milling, encapsulating, and coating.

Polypropylene pellets can be selected based on their specific melt index. For example, a melt index can be high enough such that material can be heated, softened, fused, or sintered to provide a specific pore size. The pore size can be greater than 10 microns and less than 250 microns. The pore size can be greater than 50 microns and less than 150 microns.

A melt index refers to the number of grams of a thermoplastic resin which can be forced through a 0.0825 inch orifice when subjected to 2160 grams force in 10 minutes at 190° C. Generally, a higher molecular weight will yield a lower melt index. For example, polypropylene with a molecular weight of 580,000 can have a melt index of 0.5, while a molecular weight of 174,000 can have a melt index of 2.2, and a molecular weight of 127,000 can have a melt index of 4.5. Polypropylene for use in a orthopedic implant can have a melt index high enough to allow that material to be heated, softened, fused, or sintered to a specified pore size. Generally, a higher melt index will result in a smaller pore size. For example, if a pore size between 50 and 150 microns is desired, an appropriate melt index can extrapolated from existing data, or determined experimentally, choosing polypropylene materials with increasing melt indexes until a desire pore size is reached. A polypropylene material can be selected based on its melt index, based on any desired pore size. For example, the melt index can be greater than 0.5 and less than 50. Pore size refers to the size of the holes or voids between powder particles or pellets, often measured by mercury porosimetry on open pores.

Besides the size of the original particles, the porosity of the sintered material can also be controlled by using blends of high and low melt flow materials. In some cases, high melt polymers can determine the average pore size, while the low melt polymer can give the material enhanced structural strength. Besides using blends of similar and dissimilar polymers with high and low melt flow rate, it is also possible to add other types of particulate materials in the matrix that impart other properties to the implant structure

A softening point, or the Vicat Softening Point, refers to the temperature at which a flat-ended needle of 1 square millimeter circular or square cross section will penetrate a thermoplastic specimen to a depth of 1 mm under a specified load using a uniform rate of temperature rise. (ASTM D-1525-58T).

The pellets can be molded to form a single layer or more than one layer. By coating the first layer with additional pellets and likewise subjecting the second layer to heat or pressure, a second layer can be molded and bonded to the first layer.

Suitable molds are commercially available. Suitable mold materials include, but are not limited to, metal alloys such as aluminum and stainless steel, high temperature materials, and other materials known in the art. Specific molds can have varying heights and diameters.

A spacer disc made of porous polypropylene. The pores can range in size depending on, for example, the degree of flexibility or strength desired. The pore sizes can be controlled, for example by a selected temperature, pressure, exposure time, or a combination of the above. The orthopedic implant can have similarly sized particles or blends of particle sizes of polypropylene, as is described in U.S. Pat. No. 6,083,618, which is incorporated by reference herein. The particle size distribution can be determined by commercially available screens. The particles can include polypropylene alone or polypropylene blended with other materials, other polymers, or other functional additives. The particles can be monodisperse, which can result in pore sizes having a nearly identical size and a narrow size distribution. The particles can also be polydisperse, which can result in pore sizes with a wider size distribution.

There are several methods for making porous substrates, such as sintering, using blowing agents, reverse phase precipitation, and microcell formation, such as those described by U.S. Pat. Nos. 4,473,665 and 5,160,674, which are incorporated by reference herein.

A spacer disc can be formed from particles or pellets that are monodisperse, having a narrow size distribution. The disc can also be made from particles or pellets that are polydisperse, having a wider size distribution. In one embodiment, the size distribution of the composite material particles can also be about one order of magnitude or more (expressed in micrometers). Thus, for example, if the average particle size of the composite material particles is about 20 micrometers, the composite particles can range in size from, for example, about 0.1 to about 50 micrometers. This can promote good packing of the particles and can contribute to a particularly preferred fast-hardening effect.

An orthopedic implant can be selected for its desired biocompatibility, strength, flexibility, and resistance to degradation. The orthopedic implant can also avoid undesirable reactions such as, but not limited to, thrombus formation, tumor formation, allergic reactions, and inflammation. A orthopedic implant can maintain its physical properties during the time that is remains implanted in the intervertebral space.

An orthopedic implant can include a functional additive. Functional additives are materials that contain functional groups such as, but not limited to, hydroxyl, carboxylic acid, anhydride, acyl halide, alkyl halide, aldehyde, alkene, amide, amine, guanidine, malemide, thiol, sulfonate, sulfonic acid, sulfonyl ester, carbodiimide, ester, cyano, epoxide, proline, disulfide, imidazole, imide, imine, isocyanate, isothiocyanate, nitro, or azide. Functional additives can confer additional properties to enhance the polymer's performance as a biomaterial.

Preferred functional additives are inexpensive, can be easily incorporated into the implant without degrading or losing functionality when subjected to heat or pressure or once implanted.

A orthopedic implant can include a synthetic coating or a biological coating. The coating can enhance the performance of the orthopedic implant, for example, by increasing mechanical strength, promoting cell growth, promoting biomolecule immobilization, increasing resistance to infection, improving lubricity, improving anti-thrombogenicity, and promoting stem cell or osteoblast differentiation. The coating can also allow precise biomolecular interactions to be initiated or modulated. Such coatings are commercially available, for example, from AFFINERGY™.

The coating can be applied on any surface of the orthopedic implant, such as an external surface or between polymeric layers within the implant. Biological coatings can include, for example, cell receptors, growth factors, chondrocytes, proteins, enzymes, and antibodies. Synthetic coatings can include for example, titanium, stainless steel, TEFLON®, LATEX®, collagen, PET, PETG, PGA, polystyrene, polycarbonate, glass, or nylon.

The coating can also include a biomaterial that contains modular surfaces, such as two functional peptides that can bind to a bioactive material and to a synthetic material. The two peptides can be joined by a linker that can provide cross-linking or cleaving capabilities. Interorthopedic biomaterials are commercially available, for example, from AFFINERGY™.

In one example, a orthopedic implant can include a coating, which is designed to specifically recruit and anchor osteoblasts to the implant surface. The coating can further induce differentiation of osteoblasts into bone cells using immobilized growth factors, such as BMP-2 or BMP-7. The coating can further immobilize stem cells and promote stem cell differentiation into a desired cell type, such as mineralized bone. The coating can also minimize or prevent the attachment of undesirable cells and bacteria to the implant.

A orthopedic implant can include a metal ring, such as a titanium ring, within the spacer disc to allow the disc to be viewed by x-ray or radiography techniques. The ring can also be positioned on any surface of the orthopedic implant, including top and bottom surfaces of the disc, or in between polymeric layers of the disc.

The relative amounts of polymer and additive used can vary with the specific materials used, the desired strength and flexibility of the disc, and the properties conferred by a selected additive. Depending on the desired sized and shape of the final product, this can be accomplished using a mold or a belt line disclosed by U.S. Pat. No. 3,405,206, which is incorporated by reference herein.

A orthopedic implant can be customized. For example, a customized implant can be designed based on 3-dimensional CT scan models, MRI scan models, or laser scan models, to make the implant patient-specific. The implant can also be customized by shaping, shaving, trimming, or burring the implant according to a desired shape. A burred shape refers to a sculpted or customized shape. The implant may also be modified according to additional materials or grafts that may be involved in a surgical procedure.

A orthopedic implant may be molded to have a uniform height, or a varying height. In one embodiment, a orthopedic implant can have a varying height, where the maximum height tapers to at least one edge of the implant.

Other embodiments are within the scope of the following claims.

What is claimed is:

1. An orthopedic implant comprising:
a first endplate having a tapered thickness;
a second endplate; and
a spacer disc positioned between the two endplates.

2. The implant of claim 1, wherein the second endplate has a tapered thickness.

3. The implant of claim 1, wherein the spacer disc includes at least one projection.

4. The implant of claim 1, wherein at least one of the endplates has a rounded edge.

5. The implant of claim 3, wherein the first endplate includes a depression corresponding to at least one projection on the spacer disc.

6. The implant of claim 4, wherein the second endplate includes a depression corresponding to at least one projection on the spacer disc.

7. The implant of claim 1, wherein the tapered thickness includes at least one medial thickness and at least one lateral thickness.

8. The implant of claim 7, wherein the medial thickness is less than 2 mm.

9. The implant of claim 7, wherein the medial thickness is less than 4 mm.

10. The implant of claim 7, wherein the medial thickness is less than 6 mm.

11. The implant of claim 7, wherein the lateral thickness is greater than 3 mm

12. The implant of claim 7, wherein the lateral thickness is greater than 5 mm.

13. The implant of claim 7, wherein the lateral thickness is greater than 7 mm.

14. The implant of claim 7, wherein the first or second endplate includes a cobalt chromium alloy.

15. The implant of claim 1, wherein the first endplate has an exterior surface that includes a fixation element.

16. The implant of claim 1, wherein the second endplate has an exterior surface that includes a fixation element.

17. The implant of claim 1, wherein the first or second endplate includes a fixation element that corresponds to a cortical area of a vertebra.

18. The implant of claim 1, wherein the first or second endplate includes a fixation element that corresponds to a cancellous area of a vertebra.

19. The implant of claim 1, wherein the first and second endplates have a concave interior surface.

20. The implant of claim 1, wherein the implant has an elliptical shape.

21. The implant of claim 1, wherein the first or second endplates including a coating.

22. The implant of claim 1, wherein the first or second endplates include a hydroxyapatite coating.

23. The implant of claim 1, wherein the disc is molded to a volume having a contoured shape.

24. The implant of claim 1, wherein the disc has a medial height that tapers toward a lateral edge of the disc.

25. The implant of claim 1, wherein the disc has a medial height greater than 15 mm.

26. The implant of claim 1, wherein the disc has a medial height greater than 10 mm.

27. The implant of claim 1, wherein the disc has a lateral height greater than 7 mm.

28. The implant of claim 1, wherein the disc has a lateral height greater than 2 mm.

29. The implant of claim 1, wherein the spacer disc is molded to a volume having rounded edges.

30. The implant of claim 1, wherein the spacer disc includes a metal ring.

31. The implant of claim 1, wherein the spacer disc includes polypropylene.

32. The implant of claim 1, wherein at least one of the endplates includes a cobalt chromium alloy.

33. The implant of claim 1, wherein at least one of the endplates includes titanium.

34. An orthopedic implant comprising:
a disc having at least two thickness values conforming to a distance between a first endplate and a second endplate, the disc including a metal ring;
a first endplate having a first tapered thickness; and
a second endplate having a second tapered thickness.

35. The implant of claim 34, wherein the disc includes a volume having rounded edges.

36. The implant of claim 34, wherein the disc includes polypropylene.

37. The implant of claim 34, wherein at least one of the endplates includes a cobalt chromium alloy.

38. The implant of claim 34, wherein at least one of the endplates includes titanium.

39. The implant of claim 34, wherein at least one of the endplates has a rounded edge.

40. The implant of claim 34, wherein the disc includes at least one projection.

41. The implant of claim 34, wherein the first endplate includes a depression corresponding to at least one projection on the spacer disc.

42. The implant of claim 34, wherein the second endplate includes a depression corresponding to at least one projection on the spacer disc.

43. An orthopedic implant comprising:
a first endplate having a fixation element on an exterior surface and a concave interior surface:
a second endplate having a fixation element on an exterior surface and a concave interior surface: and
a convex polypropylene disc molded to a volume that conforms to the space between the interior surfaces of the first and second endplates.

44. The implant of claim 43, wherein the disc includes a volume having rounded edges.

45. The implant of claim 43, wherein the disc includes polypropylene.

46. The implant of claim 43, wherein at least one of the endplates includes a cobalt chromium alloy.

47. The implant of claim 43, wherein at least one of the endplates has a rounded edge.

48. The implant of claim 43, wherein the disc includes at least one projection.

49. The implant of claim 43, wherein the first endplate includes a depression corresponding to at least one projection on the spacer disc.

50. The implant of claim 43, wherein the second endplate includes a depression corresponding to at least one projection on the spacer disc.

51. A method of manufacturing an orthopedic polypropylene implant including obtaining a disc, positioning the disc on a first endplate, and positioning a second endplate over the disc.

52. The method of claim 51, further comprising molding the disc to a volume having rounded edges.

53. The method of claim 51, wherein the disc includes polypropylene.

54. The method of claim 51, wherein at least one of the endplates includes a cobalt chromium alloy.

55. The method of claim 51, wherein at least one of the endplates includes titanium.

56. The method of claim 51, wherein the spacer disc includes at least one projection.

57. The method of claim 51, wherein the first endplate includes a depression corresponding to at least one projection on the spacer disc.

58. The implant of claim 51, wherein the second endplate includes a depression corresponding to at least one projection on the spacer disc.

59. A method of placing a surgical implant in a subject comprising:
obtaining an orthopedic implant;
contacting a first endplate with a natural surface of a first vertebra; and
contacting a second endplate with a natural surface of a second vertebra, the first endplate and the second endplate being separated by a spacer disc.

60. The implant of claim 59, further comprising molding the disc to a volume having rounded edges.

61. The implant of claim 59, wherein the disc includes polypropylene.

62. The implant of claim 59, wherein at least one of the endplates includes a cobalt chromium alloy.

63. The implant of claim 59, wherein at least one of the endplates includes titanium.

64. The method of claim 59, wherein the spacer disc includes at least one projection.

65. The method of claim 59, wherein the first endplate includes a depression corresponding to at least one projection on the spacer disc.

66. The implant of claim 59, wherein the second endplate includes a depression corresponding to at least one projection on the spacer disc.
